(19) United States
(12) Patent Application Publication
(10) Pub. No.: US 2024/0057948 A1
(43) Pub. Date: Feb. 22, 2024

(54) REORIENTABLE PATIENT MONITORING SYSTEM INCLUDING MULTIPLE MONITORS

(71) Applicant: DRÄGERWERK AG & CO. KGAA, Lübeck (DE)

(72) Inventors: Peter A. LUND, Nashua, NH (US); Ricardo FERNANDEZ, Beverly, MA (US)

(21) Appl. No.: 18/271,426

(22) PCT Filed: Dec. 16, 2021
(86) PCT No.: PCT/EP2021/086326

§ 371 Date: Jul. 7, 2023

Related U.S. Application Data
(60) Provisional application No. 63/131,159, filed on Dec. 28, 2020.

(51) Int. Cl. A61B 5/00

(52) U.S. Cl. CPC A61B 5/7445; A61B 2560/0431; A61B 2560/0456

(57) ABSTRACT

A patient monitoring system is disclosed and may include a monitor mount. Further, the system may include a first monitor that may have a first user interface and a plurality of ports for receiving a plurality of cables from a plurality of physiologic sensors. The system may also include a second monitor that may include a second user display. The second monitor is adapted to receive and engage the first monitor to form a dual monitor assembly and the monitor mount is adapted to receive the dual monitor assembly in a first orientation in which the plurality of cables protrude from a first side of the dual monitor assembly and a second orientation opposite the first orientation in which the plurality of cables protrude from a second side of the dual monitor assembly.

### RELATED APPLICATIONS

This document claims the priority of U.S. Application Ser. No. 63/131,159, filed Dec. 28, 2020, the content of which is hereby incorporated by reference for all purposes, including the right for priority, as if set forth verbatim herein.

### FIELD

The present disclosure generally relates to a patient monitor system having a mount, a smaller monitor, and a larger monitor that is adapted to removably receive the smaller monitor, with the smaller monitor and/or the larger monitor configured to be removably attached to the mount alone or in combination, and to provide for data and power transfer between the monitors and the mount.

### BACKGROUND

Monitors that include electronic visual displays are utilized in a large number of applications within a wide variety of industries including, for example, the healthcare industry, the military, and the oil and gas industry. Many of the applications within such industries require such monitors to, at times, be portable, and, at other times, be stationary. For example, in the healthcare industry, when not being used in transport of a patient or when a patient is ambulatory, monitors can be connected to a monitor mount. Such monitor mounts can provide a variety of functions including providing physical support, a power source, and a conduit to one or more computer networks.

One type of monitor is a patient monitor which is used by healthcare facilities to monitor and display information about a patient, such as vital signs, status of connected devices (e.g., physiological sensors, etc.), and the like. Patient monitors can be portable devices that travel with the patient in order to provide continuous monitoring during care. When a patient arrives at a hospital room or other treatment location, the patient monitor is often plugged into or otherwise connected to a patient monitor mount. Patient monitor mounts provide a physical interface for the patient monitor and are generally fixed to the treatment location. Patient monitor mounts can also provide electrical connection to other devices or infrastructure, such as power to recharge patient monitor batteries, network connectivity to other medical devices or hospital computer systems, and the like.

During the course of providing healthcare to patients, practitioners typically connect at least one type of sensor to a patient to sense, derive or otherwise monitor at least one type of patient medical parameter. Such patient connected sensors are further connected to the monitor that includes all relevant electronic components that enable conversion, manipulation and processing of the data sensed by the at least one type of sensor in order to generate patient medical parameters. These patient medical parameters may be stored in one or more modules and are usable by healthcare practitioners (e.g., nurses, doctors, physician assistants, or any other person charged with providing a healthcare service to a patient) in monitoring a patient and determining a course of healthcare to be provided to the patient. Additionally, or alternatively, the one or more modules may contain data, such as patient treatment data, to be transferred to the monitor mount and/or the monitor.

The monitor may be selectively connected to a patient at any point during which a healthcare professional comes into contact with the patient and may remain connected with the patient as the patient moves through various locations within a particular healthcare enterprise (e.g., hospital) or between different healthcare enterprises (e.g., an ambulance and/or different medical facilities). The monitor and/or the module can allow data representing the at least one patient medical parameter to be communicated to other systems within the healthcare enterprise. This data may then be used by different systems in further patient care.

In view of the foregoing, there is a need for a modular system in which a monitor assembly may be moved between at least two orientations without removing any physiologic sensor cables from the monitor assembly.

### SUMMARY

In a first aspect of the present disclosure, a patient monitoring system is disclosed and may include a monitor mount. Further, the system may include a first monitor that may have a first user interface and a plurality of ports for receiving a plurality of cables from a plurality of physiologic sensors. The system may also include a second monitor that may include a second user display. The second monitor is adapted to receive and engage the first monitor to form a dual monitor assembly and the monitor mount is adapted to receive the dual monitor assembly in a first orientation in which the plurality of cables protrude from a first side of the dual monitor assembly and a second orientation opposite the first orientation in which the plurality of cables protrude from a second side of the dual monitor assembly.

In another aspect of the present disclosure, a patient monitor is disclosed and may include a housing having a docking slot formed therein. The docking slot may be sized and shaped to receive another patient monitor therein. The patient monitor may include a handle assembly that may extend from the housing. The handle assembly may include a first handle and a second handle that may form a base adapted to support the patient monitor in an upright position.

In yet another aspect of the present disclosure, a method of operating a patient monitoring system is disclosed and may include engaging a first monitor with a second monitor to form a dual monitor assembly. A plurality of physiologic sensor cables may extend from the first monitor. The method may further include engaging the dual monitor assembly with a monitor mount in a first orientation, disengaging the dual monitor assembly for the first orientation, and engaging the dual monitor assembly with the monitor mount in a second orientation opposite the first orientation.

### BRIEF DESCRIPTION OF THE DRAWINGS

Embodiments are described herein making reference to the appended drawings.

FIG. 1 is a logical diagram illustrating a patient monitoring system (including a first monitor and a second monitor) according to an embodiment.

FIG. 2 is a front perspective view of a second monitor according to an embodiment.

FIG. 3 is a rear perspective view of a second monitor according to an embodiment.

FIG. 4 is a front perspective view of a second monitor according to another embodiment.

FIG. 5 is a front perspective view of a second monitor according to yet another embodiment.

FIG. 6 is a front perspective view of a first monitor according to an embodiment.

FIG. 7 is a rear perspective view of a first monitor according to an embodiment.

FIG. 8 is a rear perspective view of a patient monitoring system according to an embodiment.

FIG. 9 is a side view of a patient monitoring system according to an embodiment.

FIG. 10A through 10C illustrate the steps of engaging a first monitor with a second monitor to form dual monitor assembly according to an embodiment.

FIG. 11A through 11E illustrate the steps of engaging a dual monitor assembly with a monitor mount in two different orientations according to an embodiment.

### DETAILED DESCRIPTION

The following detailed description is made with reference to the accompanying drawings and is provided to assist in a comprehensive understanding of various example embodiments of the present disclosure. The following description includes various details to assist in that understanding, but these are to be regarded as merely examples. Accordingly, those of ordinary skill in the art will recognize that various changes and modifications of the examples described herein can be made without departing from the spirit and scope of the present disclosure. In addition, descriptions of well-known functions and constructions may be omitted for clarity and conciseness.

For example, in the description of the figures that follow, the automatic power on apparatus, system, method, and circuit are implemented in patient monitors. However, it should be understood and appreciated by one of ordinary skill in the art that the automatic power on apparatus, system, method, and circuit of the present disclosure can be implemented in other medical or electronic devices. The implementation of the automatic power on apparatus, system, method, and circuit in the patient monitors is meant only to assist in the understanding of the present disclosure and in no way is meant to limit the implementation the automatic power on apparatus, system, method, and circuit described herein.

Additionally, the terms and words used in the following description and claims are not limited to the bibliographical meanings, but are merely used to enable a clear and consistent understanding of the present disclosure. Accordingly, it should be apparent to those skilled in the art that the following description of the present disclosure is provided for illustration purposes only, and not for the purpose of limiting the present disclosure as defined by the appended claims and their equivalents.

Features from different embodiments may be combined to form further embodiments, unless specifically noted otherwise. Variations or modifications described with respect to one of the embodiments may also be applicable to other embodiments. In some instances, well-known structures and devices are shown in block diagram form rather than in detail in order to avoid obscuring the embodiments.

Further, equivalent or like elements or elements with equivalent or like functionality are denoted in the following description with equivalent or like reference numerals. As the same or functionally equivalent elements are given the same reference numbers in the figures, a repeated description for elements provided with the same reference numbers may be omitted. Hence, descriptions provided for elements having the same or like reference numbers are mutually exchangeable.

It is to be understood that the singular forms “a”, “an”, and “the”, include plural referents unless the context clearly dictates otherwise. Thus, for example, reference to “a processor” or “a memory” includes reference to one or more of such processors or memories.

The expressions such as “include” and “may include” which may be used in the present disclosure denote the presence of the disclosed functions, operations, and constituent elements, and do not limit the presence of one or more additional functions, operations, and constituent elements. In the present disclosure, terms such as “include” and/or “have”, may be construed to denote a certain characteristic, number, operation, constituent element, component or a combination thereof, but should not be construed to exclude the existence of or a possibility of the addition of one or more other characteristics, numbers, operations, constituent elements, components or combinations thereof.

In the present disclosure, the expression “and/or” includes any and all combinations of the associated listed words. For example, the expression “A and/or B” may include A, may include B, or may include both A and B.

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

The subject matter described herein is directed to systems and apparatuses directed to monitors (e.g., display monitors having visual electronic displays) and monitor mounts providing physical support and, in some cases, power and access to a communications/computer network. Use of such systems and apparatuses can, for example, occur in a medical environment such as the scene of a medical event, an ambulance, a hospital or a doctor's office. When a patient undergoes initial patient monitoring in such an environment, a minimum set of sensors can be connected to a patient to collect various types of patient information (e.g., physiological information) as described in detail herein. As a patient is moved from one area of care within the medical environment to another area of care, the patient monitor can travel with the patient. In some situations, the patient monitor can be mounted to a monitor mount to provide for stationary observation of the patient information on a visual electronic display. During the course of patient monitoring, the number of sensors can also increase due to increased testing and/or monitoring of the patient. In such a scenario, a patient monitor initially monitoring the patient can be docked onto a monitor mount having a second, larger monitor in order to expand the number of sensors available for patient monitoring and/or increase the number of patient parameters on a single visual electronic display by docking the smaller patient monitor to or within a larger patient monitor. The initial patient monitor can either remain within the larger patient monitor or be removed from the larger patient monitor.

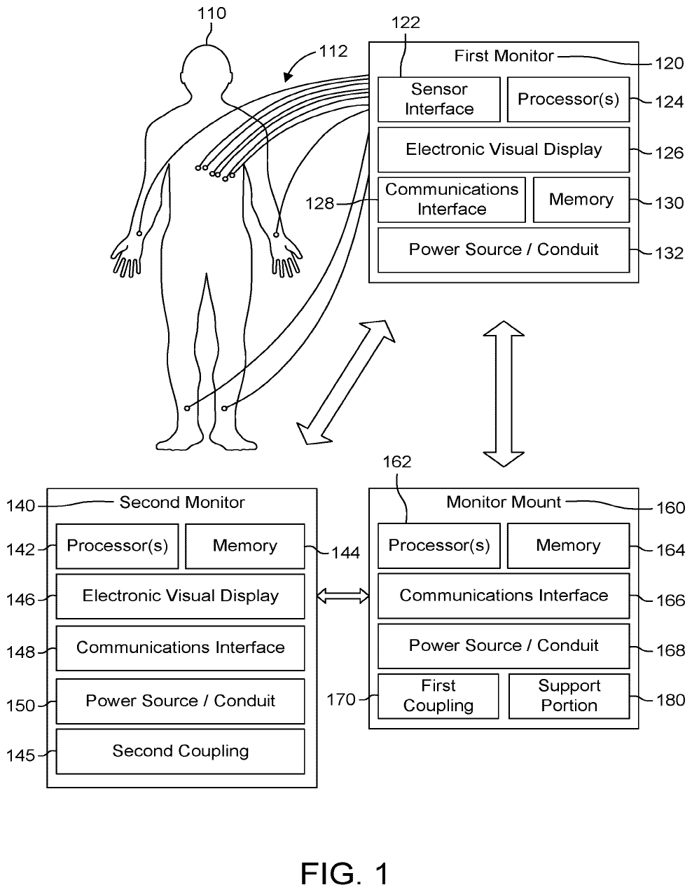

FIG. 1 is a logical diagram illustrating the example system including a first monitor 120, a second monitor 140, and a monitor mount 160. In an exemplary implementation, the monitor mount 160 may be detachably secured to a support structure (not shown) (e.g., a wall-mounted arm) via any attachment mechanism (not shown) such as a Video Electronics Standards Association (VESA) mounting interface adapted to an attachment mechanism in a hospital room in which a patient 110 is being monitored and/or treated via one or more modules, for example one or more physiological sensors and/or medical devices (connected to lead-lines 112).

The monitor mount 160 may detachably secure the second monitor 140, and the second monitor 140 can detachably secure (or otherwise physically interface with) the first monitor 120. The first monitor 120 may be first mounted or otherwise detachably secured to the second monitor 140 to form a two monitor system, and the monitor system may be then detectably secured to the monitor mount 160. Alternatively, the second monitor 140 may be first mounted or otherwise detachably secured to the monitor mount 160, and the first monitor 120 may be subsequently inserted into or otherwise mounted within a mounting area defined by the second monitor 140, the monitor mount 160, or both the second monitor 140 and the monitor mount 160. For example, the mounting area may be defined by a volume or cavity formed when the second monitor 140 and the monitor mount 160 are coupled together.

Therefore, the example system provides an interconnected, versatile, and comprehensive patient care solution with a high degree of configurability. The example system acquires data at the bedside and on transport, without having to disconnect a patient as he or she is moved from care area to care area. The example system can be scaled depending on the patient's changing acuity level and medical devices can be customized to better suit hospital protocols and use models. Accordingly, the example system thereby improves clinical workflow.

The monitor mount 160 can detachably secure (or otherwise physically interface with) both of the first monitor 120 and the second monitor 140, alone or in combination. As will be described in further detail below, the first monitor 120 has a shape and size which differs from that of the second monitor 140. Nonetheless, both of the first monitor 120 and the second monitor 140 are able to be concurrently secured to the monitor mount 160.

The first monitor 120 can, for example, be a patient monitor that is used to monitor various physiological parameters for a patient 110. With such a variation, the first monitor 120 can include a sensor interface 122 that can be used to connect via wired and/or wireless interfaces to one or more physiological sensors and/or medical devices (e.g., such as those attached to lead-lines 112 that may include, for example, electrocardiogram (ECG) electrodes, an oxygen saturation (SpO2) sensor, noninvasive blood pressure (NIBP) sensors, blood pressure cuffs, apnea detection sensors, end-tidal carbon dioxide (etCO2) sensors, respirators, temperature, and other similar physiological data.) associated with the patient 110. The first monitor 120 can include one or more processors 124 (e.g., programmable data processors, etc.) which can execute various instructions stored in memory 130 of the first monitor 120. Various data and graphical user interfaces can be conveyed to a user via an electronic visual display 126 included in the first monitor 120. This information can, for example, relate to the measured physiological parameters of the patient 110 and the like (e.g., ECG waveforms, blood pressure, heart related information, pulse oximetry, respiration information, temperature, etc.). Other types of information can also be conveyed by the electronic visual display 126. In some variations, the electronic visual display 126 includes a touch screen interface that allows a user of the first monitor 120 to input data and/or modify the operation of the first monitor 120.

The first monitor 120 can additionally include a communications interface 128 which allows the first monitor 120 to directly or indirectly (via, for example, the monitor mount 160) access one or more computing networks. The communications interface 128 can include, various network cards/interfaces to enable wired and wireless communications with such computing networks. The communications interface 128 can also enable direct (i.e., device-to-device, etc.) communications (i.e., messaging, signal exchange, etc.) such as from the monitor mount 160 to the first monitor 120.

The first monitor 120 can optionally also include a power source and/or conduit 132 that can be used to power the various components of the first monitor 120 (and optionally various components of the second monitor 140 and/or the monitor mount 160). The power source/conduit 132 can include a self-contained power source such as a battery pack and/or the power source/conduit 132 can include an interface to be powered through an electrical outlet (either directly or indirectly by way of the second monitor 140 and/or the monitor mount 160). In some variations, the first monitor 120 can only be powered and render information when secured or otherwise connected to one or more of the second monitor 140 and the monitor mount 160.

The second monitor 140 can include one or more processors 142 (e.g., programmable data processors, etc.) which can execute various instructions stored in memory 144 of the second monitor 140. Various data and graphical user interfaces can be conveyed to the user via an electronic visual display 146 included in the second monitor 140. This information can, for example, relate to the measured physiological parameters of the patient 110 and the like (e.g., blood pressure, heart related information, pulse oximetry, respiration information, thermoregulation, neonatal information, ventilator information, anesthesia information, incubation information, etc.) as received from the first monitor 120. Other types of information can also be conveyed by the electronic visual display 146. In some variations, the electronic visual display 146 includes a touch screen interface that allows a user of the second monitor 140 to input data and/or modify the operation of the second monitor 140.

The second monitor 140 can additionally include a communications interface 148 which allows the second monitor 140 to directly or indirectly (via, for example, the first monitor 120 and/or the monitor mount 160) access one or more computing networks. The communications interface 148 can include various network cards/interfaces to enable wired and wireless communications with such computing networks. The communications interface 148 can also enable direct (i.e., device-to-device, etc.) communications (i.e., messaging, signal exchange, etc.) such as from the monitor mount 160 to the second monitor 140 and the first monitor 120 to the second monitor 140.

The second monitor 140 can optionally also include a power source and/or conduit 150 that can be used to power the various components of the second monitor 140 (and optionally various components of the first monitor 120). The power source/conduit 150 can include a self-contained power source such as a battery pack and/or the power source/conduit 150 can include an interface to be powered through an electrical outlet (either directly or by way of the first monitor 120 and/or the monitor mount 160). In some variations, the second monitor 140 can only be powered and render information when secured or otherwise connected to one or more of the first monitor 120 and the monitor mount 160.

The second monitor 140 can include a second coupling 145 which is configured to detachably secure the first monitor 120. In some variations, the second coupling 145 may be positioned in a receptacle of the second monitor 140. The receptacle may be defined in a lateral direction of the second monitor 140 and have open side portions for receiving the first monitor 120. For example, the user can visually confirm the location of the second coupling 145 and transversely insert the first monitor 120 into the second monitor 140. In some variations, the receptacle may have an open top portion instead of open side portions such that the first monitor 120 can be dropped into the second monitor 140 from above; and removed (e.g., lifted out) from the second monitor 140 from above.

The monitor mount 160 can include one or more processors 162 (e.g., programmable data processors, etc.) which can execute various instructions stored in memory 164 of the monitor mount 160. The monitor mount 160 can additionally include a communications interface 166 which allows the monitor mount 160 to directly or indirectly access one or more computing networks. The communications interface 166 can include various network cards/interfaces to enable wired and wireless communications with such computing networks. The communications interface 166 can also enable direct (i.e., device-to-device, etc.) communications (i.e., messaging, signal exchange, etc.) such as with the first monitor 120 and/or the second monitor 140.

The monitor mount 160 can optionally also include a power source and/or conduit 168 that can be used to power the various components of the monitor mount 160 and/or the first monitor 120 and/or the second monitor 140 when secured to the monitor mount 160. The power source/conduit 168 can include a self-contained power source such as a battery pack and/or the power source/conduit 168 can include an interface to be powered through an electrical outlet.

Any of the processors 124, 142, 162 may acquire data from any of the monitor mount 160 and one or more of the monitors 120, 140 and store the acquired data in a memory and, upon connection of the monitor mount 160 and one or more of the monitors 120, 140, transfer the data stored in the memory to the monitor mount 160 or one or more of the monitors 120, 140. The data may include any of patient identification data including information identifying a patient; patient parameter data representing at least one type of patient parameter being monitored; and device configuration data including information associated with configuration settings for the monitor mount 160 and/or the one or more monitors 120, 140.

The monitor mount 160 can optionally also include any mounting interface, such as a VESA mounting interface for mounting the monitor mount at the bedside, from the ceiling, on a wall of the room, or even outside the room for isolation purposes.

The monitor mount 160 can optionally also include an interface configured to receive a connector of a cable or wired connection for connecting a module, a monitor, other external unit or the like.

The monitor mount 160 can optionally also include one or more recesses for facilitating removal of the first monitor 120 and/or the second monitor 140.

In some variations, the one or more processors 162 and the memory 164 are omitted such that the monitor mount 160 provides only physical support and optionally a power source.

The monitor mount 160 has a shape and size which allows the monitor mount 160 to detachably secure both of the first monitor 120 and the second monitor 140 such that the respective monitors 120 and 140 can be removed by the user when desired.

The monitor mount 160 can include a first coupling 170 to allow the first monitor 120 and/or second monitor 140 to be secured to the monitor mount 160. The monitor mount 160 is able to secure each of the first monitor 120 and the second monitor 140 individually or both of the first monitor 120 and the second monitor 140 concurrently. In other words, the first coupling 170 is configured to accept either the first monitor 120 or the second monitor 140 such that the monitor mount 160 is configured to mount the first monitor 120 alone, the second monitor 140 alone, or a combination of the first monitor 120 and the second monitor 140. The first coupling 170 can include any mechanical attachment means such as a ledge, a rail, a rib, an abutment, a cleat, and the like, or any combination thereof.

The first coupling 170 can additionally or alternatively include different securing mechanisms including magnetic and/or electromagnetic locking mechanisms which cause the first monitor 120 to selectively be secured to the monitor mount 160. In some cases, the first monitor 120 can slide into and out of the first coupling 170 from one or more lateral directions (i.e., from one or more sides of the monitor mount 160) while in other variations, the first monitor 120 can be mounted to and removed from the front face of the monitor mount 160. In some implementations, the first monitor 120 can both slide into and out of the first coupling 170 from one or more lateral directions and be mounted to and removed from the front face of the monitor mount 160.

The positioning of the first monitor 120 when secured to the monitor mount 160 can be such that the communications interface 128 on the first monitor 120 aligns with the communications interface 166 of the monitor mount 160 to allow, for example, a direct connection (e.g., electrical connection). In other variations, the communications interface 128 of the first monitor 120 exchanges data with the communications interface 166 of the monitor mount 160 wirelessly (via, for example, optical communication by way of respective optical windows on the first monitor 120 and the monitor mount 160). For example, both communication interfaces 128 and 166 may include bi-directional phototransceivers that are configured for bi-directional communication. The communications interface 128 of the first monitor 120 may be located on a back facing portion of the first monitor 120, whereas the communications interface 166 may be located on a front facing portion of the monitor mount 160 so that the back facing portion and the front facing portion face each other when the first monitor 120 is mounted to the monitor mount 160.

The positioning of the first monitor 120 when secured to the monitor mount 160 can also align the power source/conduit 132 of the first monitor 120 to be coupled to the power source/conduit 168 of the monitor mount 160 which causes the monitor mount 160 to power the first monitor 120.

The monitor mount 160 can include a support portion 180 to allow the second monitor 140 to be secured to the monitor mount 160. The support portion 180 may be positioned at a top of the monitor mount 160 or a bottom of the monitor mount 160. The support portion 180 can include any mechanical attachment means such as a ledge, a rail, a rib, an abutment, a cleat, and the like, or any combination thereof. The positioning of the second monitor 140 when secured to the monitor mount 160 can be such that the communications interface 148 on the second monitor 140 aligns with the communications interface 166 of the monitor mount 160 to allow, for example, a direct connection (e.g., electrical connection). In other variations, the communications interface 148 of the second monitor 140 exchanges data with the communications interface 166 of the monitor mount 160 wirelessly (via, for example, optical communication by way of respective optical windows on the second monitor 140 and the monitor mount 160). For example, both communication interfaces 128 and 166 may include bi-directional phototransceivers that are configured for bi-directional communication. These connections (electrical, optical, wireless, etc.) can be used to sense position (docking, undocking) as well as the type of monitor mount 160 out of a plurality of types of mounts. The communications interface 148 of the second monitor 140 may be located on a back portion of the second monitor 140. The communications interface 148 of the second monitor 140 may be located on a back facing portion of the second monitor 140, whereas the communications interface 166 may be located on a front facing portion of the monitor mount 160 so that the back facing portion and the front facing portion face each other when the second monitor 140 is mounted to the monitor mount 160.

The support portion 180 can enable front-to-back docking of the second monitor 140 to the monitor mount 160 by providing a shelf or similar feature extending outwardly. This feature of the support portion 180 can support and/or disperse the weight of the second monitor 140 during positioning of the second monitor 140. For example, a user attempting to position the second monitor 140 onto the monitor mount 160 can rest the second monitor 140 on the support portion 180 during the positioning while attaching the back portion of the second monitor 140 to the first coupling 170. The support portion 180 can support a bottom face of the second monitor 140.

Alternatively, or additionally, the support portion 180 can enable hanging or suspension of a handle of the second monitor 140 from the monitor mount 160 by providing any mechanical attachment means such as a ledge, a rail, a rib, an abutment, a cleat, and the like, or any combination thereof extending laterally from the top portion of mount 160. This feature of the support portion 180 can support and/or disperse the weight of the second monitor 140 during positioning of the second monitor 140. For example, a user attempting to position the second monitor 140 to the monitor mount 160 can hang or suspend the handle of the second monitor 140 from the support portion 180 during the positioning while attaching the back portion of the second monitor 140 to the first coupling 170.

The positioning of the second monitor 140 when secured to the monitor mount 160 can also align the power source/conduit 150 of the second monitor 140 to be coupled to the power source/conduit 168 of the monitor mount 160 which causes the monitor mount 160 to power the second monitor 140 or vice-versa. In some variations, the positioning of the second monitor 140 when secured to the monitor mount 160 and/or when the first monitor 120 is also secured to the monitor mount 160 can also align the power source/conduit 150 of the second monitor 140 to be coupled to the power source/conduit 132 of the first monitor 120 (which in turn is connected to the power source/conduit 168 of the monitor mount 160) which causes the first monitor 120 to power the second monitor 140.

The modular mounting of the three devices 120, 140, and 160 will now be described in greater detail. The modular mounting may allow the first monitor 120 to dock into the monitor mount 160 from the mount's front surface, allow the first monitor 120 to dock into the monitor mount 160 by sliding the first monitor 120 in from the left and/or the right lateral side of the monitor mount 160, allow the combination of the first monitor 120 and the second monitor 140 to dock to the monitor mount 160, allow the first monitor 120 to slide out of the combination of the monitor mount 160 and the second monitor 140 while the monitor mount 160 and the second monitor 140 remain mechanically coupled to one another, allow the second monitor 140 to be mounted to the monitor mount 160 in the absence of the first monitor 120, and any combination thereof.

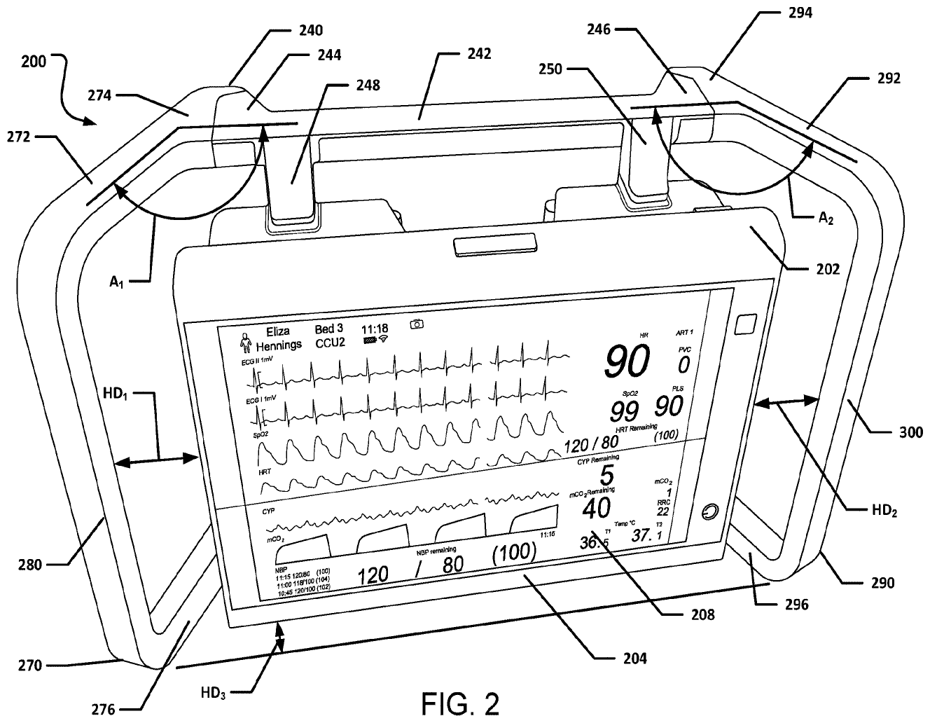

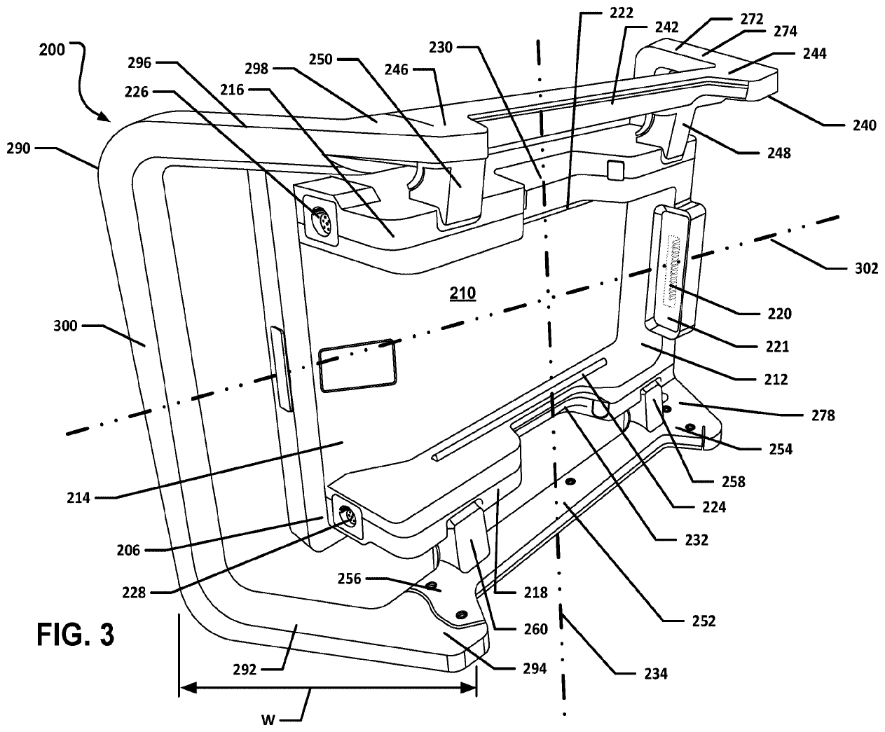

Referring now to FIG. 2 and FIG. 3, a second monitor 200 is illustrated and represents one example implementation. In a particular aspect, the second monitor 200 can be the second monitor 140 described above in conjunction with the modular system. As shown, the second monitor 200 may include a housing 202 having a front 204 (illustrated in FIG. 2 as the foreground) and a back 206 (not fully visible in FIG. 2 and largely at the rear of this perspective view). The front 204 of the housing 202 of the monitor 200 may include a user interface 208 incorporated therein. The user interface 208 may be a display, a touch display, or a combination thereof.

As depicted in FIG. 3, the back 206 of the housing 202 of the monitor 200 may include a docking slot 210 that may extend into the back 206 of the housing 202. Further, the docking slot 210 may extend at least partially along a length of the back 206 of the housing 202. FIG. 3 further shows that the docking slot 210 may include an enclosed end 212 and an open end 214. The docking slot 210 may also include a first side wall 216 that may extend from the open end 214 of the docking slot 210 to the enclosed end 212 of the docking slot 210. Further, the docking slot 210 may include a second side wall 218 opposite the first side wall 216. The second side wall 218 may extend from the open end 214 of the docking slot 210 to the enclosed end 212 of the docking slot 210. The second side wall 218 may be substantially parallel to the first side wall 216.

In a particular aspect, the enclosed end 212 of the docking slot 210 may include a docking connector 220 within a docking connector recess 221 formed in the enclosed end 212 of the docking slot 210. The docking connector recess 221 may be sized and shaped to receive a docking connector plate of a first monitor, described in detail below. As depicted, the first side wall 216 may include a first elongated protrusion 222, e.g., a rib, that may extend from the first side wall 216 into the docking slot 210. The first elongated protrusion 222 may extend from the first side wall 216 in a direction that is substantially perpendicular to the first side wall 216. As shown, a second elongated protrusion 224, e.g., a rib, may extend from the second side wall 218 into the docking slot 210. The second elongated protrusion 224 may extend from the second side wall 218 in a direction that is substantially perpendicular to the second side wall 218. In a particular aspect, the elongated protrusions 222, 224 may have a rounded, or convex, cross section. Specifically, the elongated protrusions 222, 224 may have a semi-cylindrical shape and each elongated protrusion 222, 224 may have a semi-circular shape in a cross section taken along an axis perpendicular to a longitudinal axis of each elongated protrusion 222, 224.

In a particular aspect, the docking slot 210 may be sized and shaped to receive a smaller monitor, e.g., a first monitor, described in detail below. As such, when the smaller monitor is engaged with the second monitor 200, the smaller monitor may be slid into the docking slot 210 between the first sidewall 216 and the second sidewall 218 from the open end 214 to the enclosed end 212 until a connector on the smaller monitor is engaged with the docking connector 220 formed in the enclosed end 212 of the docking slot 210. As shown, the first side wall 216 may include a first auxiliary port 226 at an end of the first side wall 216 near the open end 214 of the docking slot 210 and the second side wall 218 may include a second auxiliary port 228 at an end of the second side wall 218 near the open end 214 of the docking slot 210.

FIG. 3 also shows that the back 206 of the housing 202 of the second monitor 200 may be formed with a first latch mechanism 230 within the first sidewall 216 of the docking slot 210. The back 206 of the housing 202 of the second monitor 200 may also be formed with a second latch mechanism 232 within the second sidewall 218 of the docking slot 210. The first latch mechanism 230 and second latch mechanism 232 may be sized and shaped to receive and engage a pair of latches on a monitor mount, described below. The latch mechanisms 230, 232 may be centered along a central vertical axis 234 of the housing 202 of the second monitor 200.

Referring to FIG. 2 and FIG. 3, the second monitor 200 may include a handle assembly 240 attached to the housing 202 of the second monitor 200. The handle assembly 240 may include a first transverse member 242 that may have a first end 244 and a second end 246. Moreover, the handle assembly 240 may include a first vertical mounting post 248 that may extend from the first transverse member 242 near the first end 244 of the first transverse member 242. The first vertical mounting post 248 may extend from the first transverse member 242 in a direction that is substantially perpendicular to the first transverse member 242. The handle assembly 240 may include a second vertical mounting post 250 that may extend from the first transverse member 242 of the handle assembly 240 near the second end 246 of the first transverse member 242. The second vertical mounting post 250 may extend from the first transverse member 242 in a direction that is substantially perpendicular to the first transverse member 242 and substantially parallel to the first vertical mounting post 248.

As depicted in FIG. 3, the handle assembly 240 may further include a second transverse member 252 that may have a first end 254 and a second end 256. Moreover, the handle assembly 240 may include a third vertical mounting post 258 that may extend from the second transverse member 252 near the first end 254 of the second transverse member 252. The third vertical mounting post 258 may extend from the second transverse member 252 in a direction that is substantially perpendicular to the second transverse member 252. The handle assembly 240 may further include a fourth vertical mounting post 260 that may extend from the second transverse member 252 of the handle assembly 240 near the second end 256 of the second transverse member 252. The fourth vertical mounting post 260 may extend from the second transverse member 252 in a direction that is substantially perpendicular to the second transverse member 252 and substantially parallel to the third vertical mounting post 258.

In a particular aspect, the first vertical mounting post 248 of the handle assembly 240 may be vertically aligned with the third vertical mounting post 258 of the handle assembly 240. Also, the second vertical mounting post 250 of the handle assembly 240 may be vertically aligned with the fourth vertical mounting posts 260. The first and third vertical mounting posts 248, 258 may be spaced a first distance, D₁, away from the central vertical axis 234. The second and fourth vertical mounting posts 250, 260 may be spaced a second distance, D₂, away from the central vertical axis 234. D₁ may be substantially equal to D₂.

Referring again to FIG. 2 and FIG. 3, the handle assembly 240, may include a first handle 270 extending between the first transverse member 242 and the second transverse member 252. The first handle 270 may include a first extension 272 that may have a first end 274 and a second extension 276 that may have a second end 278 (visible in FIG. 3). The first handle 270 of the handle assembly 240 may include a first grip portion 280 extending between the first extension 272 and the second extension 276. In a particular aspect, as illustrated, the first end 274 of the first handle 270 may be connected, or otherwise coupled, to the first end 244 of the first transverse member 242. Moreover, the second end 278 of the first handle 270 may be connected, or otherwise coupled, to the first end 254 of the second transverse member 252. As shown, the extensions 272, 276 of the first handle 270 may extend in a direction generally outward and forward from the transverse members 242, 252. Further, the extensions 272, 276, and the first handle 270, may extend from the transverse members 242, 252 at and a first angle, A₁, with respect to the transverse members 242, 252.

In a particular aspect, A₁ may greater than or equal to 95°. In another aspect, A₁ may be greater than or equal to 100°, such as greater than or equal to 105°, greater than or equal to 110°, greater than or equal to 115°, greater than or equal to 120°, or greater than or equal to 125°. In still another aspect, A₁ may be less than or equal to 150°, such as less than or equal to 145°, less than or equal to 140°, less than or equal to 135°, or less than or equal to 130°. In yet another aspect, A₁ may be within a range between, and including, the minimum and maximum values of A₁ described herein.

As further indicated in FIG. 2, the handle assembly 240 is constructed so that the first grip portion 280 of the first handle 270 may be spaced a first handle distance, HD₁, from the housing 202 of the second monitor 200. It is to be appreciated that HD₁ may be measured from the inner edge of the first grip portion 280 of the first handle 270 and the outer edge of the housing 202 of the second monitor 200. In one aspect, HD₁ may be greater than or equal to 2.50 centimeters (cm). Further, HD₁ may be greater than or equal to 2.75 cm, such as greater than or equal to 3.00 cm, greater than or equal to 3.25 cm, greater than or equal to 3.50 cm, greater than or equal to 3.75 cm, or greater than or equal to 4.00 cm. In another aspect, HD₁ may be less than or equal to 6.5 cm, such as less than or equal to 6.25 cm, less than or equal to 6.00 cm, less than or equal to 5.75 cm, less than or equal to 5.50 cm, less than or equal to 5.25 cm, less than or equal to 5.00 cm, less than or equal to 4.75 cm, less than or equal to 4.50 cm, or less than or equal to 4.25 cm. In still another aspect, HD₁ may be within a range between, and including, any of the minimum and maximum values of HD₁ described herein.

The handle assembly 240, may also include a second handle 290 extending between the first transverse member 242 and the second transverse member 252. The second handle 290 may include a first extension 292 that may have a first end 294 and a second extension 296 that may have a second end 298 (visible in FIG. 3). The second handle 290 of the handle assembly 240 may include a first grip portion 300 extending between the first extension 292 and the second extension 296 of the second handle 290. In a particular aspect, as illustrated, the first end 294 of the second handle 290 may be connected, or otherwise coupled, to the second end 246 of the first transverse member 242. Moreover, the second end 298 of the second handle 290 may be connected, or otherwise coupled, to the second end 256 of the second transverse member 252. As shown, the extensions 292, 296 of the second handle 290 may extend in a direction generally outward and forward from the transverse members 242, 252. Further, the extensions 292, 296, and the second handle 290, may extend from the transverse members 242, 252 at and a first angle, A₂, with respect to the transverse members 242, 252.

In a particular aspect, A₂ may greater than or equal to 95°. In another aspect, A₂ may be greater than or equal to 100°, such as greater than or equal to 105°, greater than or equal to 110°, greater than or equal to 115°, greater than or equal to 120°, or greater than or equal to 125°. In still another aspect, A₂ may be less than or equal to 150°, such as less than or equal to 145°, less than or equal to 140°, less than or equal to 135°, or less than or equal to 130°. In yet another aspect, A₂ may be within a range between, and including, the minimum and maximum values of A₂ described herein. In another aspect, A₂ may be equal to A₁.

As further indicated in FIG. 2, the handle assembly 240 is constructed so that the second grip portion 300 of the second handle 290 may be spaced a second handle distance, HD₂, from the housing 202 of the second monitor 200. It is to be appreciated that HD₂ may be measured from the inner edge of the second grip portion 300 of the second handle 290 and the outer edge of the housing 202 of the second monitor 200. In one aspect, HD₂ may be greater than or equal to 2.50 centimeters (cm). Further, HD₂ may be greater than or equal to 2.75 cm, such as greater than or equal to 3.00 cm, greater than or equal to 3.25 cm, greater than or equal to 3.50 cm, greater than or equal to 3.75 cm, or greater than or equal to 4.00 cm. In another aspect, HD₂ may be less than or equal to 6.5 cm, such as less than or equal to 6.25 cm, less than or equal to 6.00 cm, less than or equal to 5.75 cm, less than or equal to 5.50 cm, less than or equal to 5.25 cm, less than or equal to 5.00 cm, less than or equal to 4.75 cm, less than or equal to 4.50 cm, or less than or equal to 4.25 cm. In still another aspect, HD₂ may be within a range between, and including, any of the minimum and maximum values of HD₂ described herein. In another aspect, HD₂ may be equal to HD₁.

As further illustrated in FIG. 2, the handles 270, 290, or the grips 280, 300 thereof, may extend beyond the front of the housing 202 of the second monitor 200. For example, the handles 270, 290, or the grips 280, 300 thereof, may extend a handle distance, HD₃, beyond the front of the housing 202 of the second monitor 200. In a particular aspect, HD₃ may be greater than or equal to 0.50 cm. Further, HD₃ may be greater than or equal to 0.75 cm, such as greater than 1.00 cm, greater than 1.25 cm, or greater than 1.5 cm. In another aspect, HD₃ may be less than or equal to 2.50 cm, such as less than or equal to 2.25 cm, less than or equal to 2.00 cm, or less than or equal to 1.75 cm. In yet another aspect, HD₃ may be within a range between, and including, any of the minimum and maximum values of HD₃ described herein.

It is to be understood that the second monitor 200 may be symmetrical about a central longitudinal, or horizontal, axis 302. Moreover, the handle assembly 240 may be symmetrical about the central longitudinal axis 302 and about the central vertical axis 234. Further, the first grip portion 280 of the handle assembly 240 may be substantially parallel to the second grip portion 300 of the handle assembly 240. FIG. 3 shows that the second monitor 200 may have an overall width, W, that may be measured between the front of the handle assembly 240 and the back of the handle assembly 240. In a particular aspect, W, may be greater than or equal to 7.00 cm. Further, W may be greater than or equal to 7.50 cm, such as greater than or equal to 8.00 cm, greater than or equal to 8.50 cm, or greater than 9.00 cm. In another aspect, W may be less than or equal to 12.00 cm, such as less than or equal to 11.50 cm, less than or equal to 11.00 cm, less than or equal to 10.50 cm, less than or equal to 10.00 cm, or less than or equal to 9.50 cm. In still another aspect, W may be within a range between, and including, any of the minimum and maximum values of W.

The handle assembly 240 may provide a base on which the second monitor 200 may rest on without tipping over. In other words, the handle assembly 240 may support the second monitor 200 in an upright position, either alone or when a first monitor, described below, is engaged therewith, e.g., with the docking slot 210. The grip portions 280, 300 of each respective handle 270, 290 may be spaced from the housing 202 of the second monitor 200 in order to allow a user to wrap their fingers around the grip portion. This allows the user to easily grasp the second monitor 200 and rotate the second monitor 200, and a first monitor engaged therewith, as described below, without risking dropping the second monitor 200 or the assembly of the second monitor 200 and the first monitor. Further, the handles 270, 290 are swept forward to prevent the handles 270, 290 from interfering with, kinking, or otherwise fouling, a plurality of physiologic sensor cables that may be connected to a first monitor that is engaged with the second monitor 200. The handles 270, 290 may also help guide the cables to prevent issues therewith.

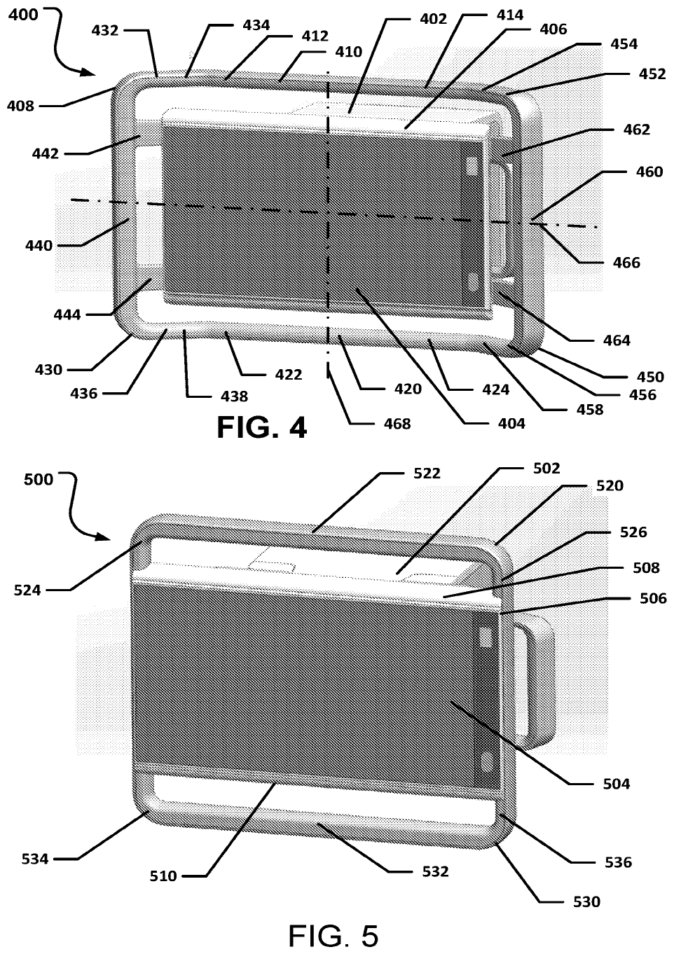

Referring now to FIG. 4, an alternative embodiment of a second monitor 400 is illustrated. As shown, the second monitor 400 may include a housing 402. A user interface 404 may be incorporated into, or otherwise disposed on, a front 406 of the housing 402. The user interface 404 may be a display, such as a touch screen display.

As illustrated, the second monitor 400 may include a handle assembly 408. The handle assembly 408 may be attached to the housing 402 of the second monitor 400 as described below. Specifically, the handle assembly 408 may include a first transverse member 410 that may have a first end 412 and a second end 414. The handle assembly 408 may also include a second transverse member 420 that may have a first end 422 and a second end 424.

The handle assembly 408 may also include a first handle 430 extending between the first transverse member 410 and the second transverse member 420. The first handle 430 may include a first extension 432 that may have a first end 434 and a second extension 436 that may have a second end 438. The first handle 430 of the handle assembly 408 may include a first grip portion 440 extending between the first extension 432 and the second extension 436.

In a particular aspect, as illustrated, the first end 434 of the first handle 430 may be connected, or otherwise coupled, to the first end 412 of the first transverse member 410. Moreover, the second end 438 of the first handle 430 may be connected, or otherwise coupled, to the first end 422 of the second transverse member 420. As shown, the extensions 432, 436 of the first handle 430 may extend in a direction generally outward and forward from the transverse members 410, 420. Further, the extensions 432, 436, and the first handle 430, may extend from the transverse members 410, 412 at and an angle with respect to the transverse members 410, 412. That angle may be the same as A₁, described above.

As shown in FIG. 4, a first horizontal mounting post 442 may extend from the first grip portion 440 of the first handle 430 near the first extension 432. The first horizontal mounting post 442 may be substantially parallel to the first extension 432. Further, the first horizontal mounting post 442 may extend from the first grip portion 440 and connect to the housing 402 of the monitor 400 at an angle. That angle may be the same as A₁, described above. A second horizontal mounting post 444 may extend from the first grip portion 440 of the first handle 430 near the second extension 436. The second horizontal mounting post 444 may be substantially parallel to the second extension 436 and the first horizontal mounting post 442. Moreover, the second horizontal mounting post 444 may extend from the first grip portion 440 and connect to the housing 402 of the monitor 400 at an angle. That angle may be the same as A₁, described above.

FIG. 4 shows that the handle assembly 408 may also include a second handle 450 extending between the first transverse member 410 and the second transverse member 420. The second handle 450 may include a first extension 452 that may have a first end 454 and a second extension 456 that may have a second end 458. The second handle 450 of the handle assembly 408 may include a second grip portion 460 extending between the first extension 452 and the second extension 456.

In a particular aspect, as illustrated, the first end 454 of the second handle 450 may be connected, or otherwise coupled, to the second end 414 of the first transverse member 410. Moreover, the second end 458 of the second handle 450 may be connected, or otherwise coupled, to the second end 424 of the second transverse member 420. As shown, the extensions 452, 456 of the second handle 450 may extend in a direction generally outward and forward from the transverse members 410, 420. Further, the extensions 452, 456, and the second handle 450, may extend from the transverse members 410, 412 at and an angle with respect to the transverse members 410, 412. That angle may be the same as A₂, described above.

As shown in FIG. 4, a third horizontal mounting post 462 may extend from the second grip portion 460 of the second handle 450 near the first extension 452. The third horizontal mounting post 462 may be substantially parallel to the first extension 452. Further, the first horizontal mounting post 462 may extend from the second grip portion 460 and connect to the housing 402 of the monitor 400 at an angle. That angle may be the same as A₂, described above. A fourth horizontal mounting post 464 may extend from the second grip portion 460 of the second handle 450 near the second extension 456. The fourth horizontal mounting post 464 may be substantially parallel to the second extension 456 and the third horizontal mounting post 462. Moreover, the fourth horizontal mounting post 464 may extend from the first grip portion 460 and connect to the housing 402 of the monitor 400 at an angle. That angle may be the same as A₂, described above.

In a particular aspect, the first horizontal mounting post 442 of the handle assembly 408 may be horizontally aligned with the third horizontal mounting post 462 of the handle assembly 408. Also, the second horizontal mounting post 444 of the handle assembly 408 may be horizontally aligned with the fourth horizontal mounting posts 464. The first and third horizontal mounting posts 442, 462 may be spaced a first distance, D₁, away from a central horizontal axis 466. The second and fourth horizontal mounting posts 462, 464 may be spaced a second distance, D₂, away from the central horizontal axis 234. D₁ may be substantially equal to D₂. Further, the handle assembly 408 may be substantially symmetrical about the central horizontal axis 466. The handle assembly 408 may also be substantially symmetrical about a central vertical axis 468. Also, the first grip portion 440 of the handle assembly 408 may be substantially parallel to the second grip portion 460 of the handle assembly 408.

As further indicated in FIG. 4, the handle assemble 430 may be constructed so that the first grip portion 440 of the first handle 430 may be spaced a handle distance from the housing 402 of the second monitor 400. It is to be appreciated that the handle distance may be measured from the inner edge of the first grip portion 440 of the first handle 430 and the outer edge of the housing 402 of the second monitor 400. The handle distance may be the same as HD₁, described above. The second grip portion 460 of the second handle 450 may be spaced a handle distance from the housing 402 of the second monitor 400. It is to be appreciated that the handle distance may be measured from the inner edge of the first grip portion 440 of the first handle 430 and the outer edge of the housing 402 of the second monitor 400. The handle distance may be the same as HD₂, described above.

As further illustrated in FIG. 4, the handles 430, 450, or the grips 440, 460 thereof, may extend beyond the front of the housing 402 of the second monitor 400. For example, the handles 430, 450, or the grip portions 440, 460 thereof, may extend a handle distance beyond the front of the housing 402 of the second monitor 400. In a particular aspect, the handle distance may be equal to HD₃, described above.

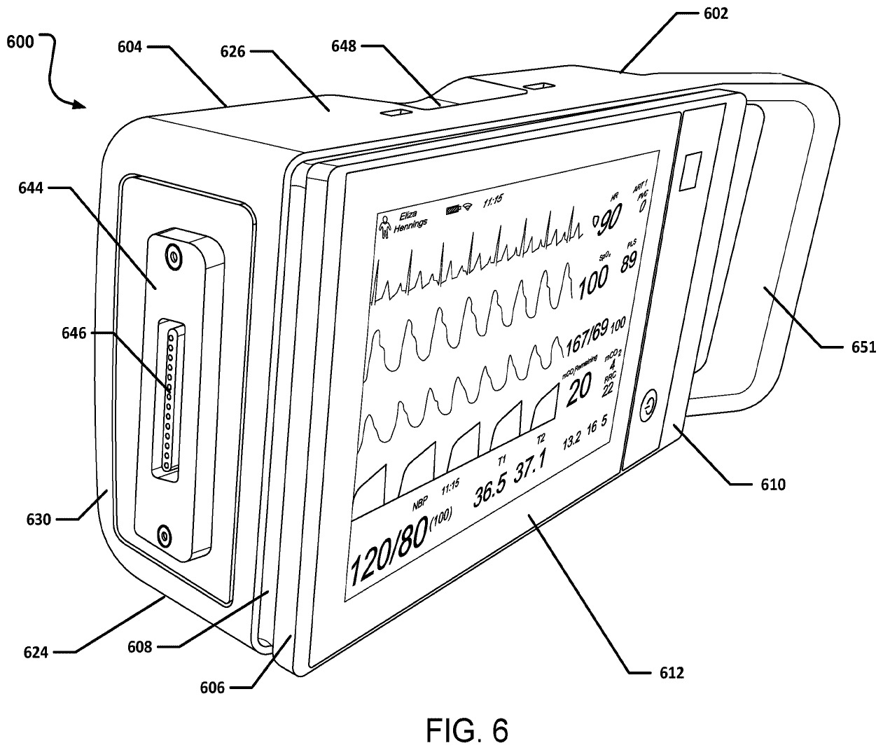

FIG. 5 depicts another alternative embodiment of a second monitor 500. As shown, the second monitor 500 may include a housing 502. A user interface 504 may be incorporated into, or otherwise disposed on, a front 506 of the housing 502. The user interface 504 may be a display, such as a touch screen display. Further, the housing 502 may include, or define, a first horizontal face 508 adjacent to the user interface 504 and a second horizontal face 510 adjacent to the user interface 504 opposite

As illustrated, the second monitor 500 may include a first handle 520 extending from the first horizontal face 508 of the housing 502 of the second monitor 500. The first handle 520 may include a first grip portion 522 that may include a first end 524 and a second end 526. As shown, the ends 524, 526 of the first grip portion 522 may be connected to the housing 502. Further, the first grip portion 522 may be substantially parallel to the first horizontal face 508 of the housing 502. The ends 524, 526 may be substantially perpendicular to the first grip portion 522 and substantially parallel to each other. The first grip portion 522 of the first handle 520 may be spaced a handle distance from the first horizontal face 508 of the housing 502 of the monitor 500. The handle distance may be measured between an inner edge of the first grip portion 522 and the first horizontal face 508 of the housing 502 of the monitor 500 and the handle distance may be the same as HD₁, described above.

The second monitor 500 may also include a second handle 530 extending from the second horizontal face 510 of the housing 502 of the second monitor 500. The second handle 530 may include a second grip portion 532 that may include a first end 534 and a second end 536. As shown, the ends 534, 536 of the second grip portion 532 may be connected to the housing 502. Further, the second grip portion 532 may be substantially parallel to the second horizontal face 510 of the housing 502. The ends 534, 536 may be substantially perpendicular to the second grip portion 532 and substantially parallel to each other. The second grip portion 532 of the second handle 530 may be spaced a handle distance from the second horizontal face 510 of the housing 502 of the monitor 500. The handle distance may be measured between an inner edge of the second grip portion 532 and the second horizontal face 510 of the housing 502 of the monitor 500 and the handle distance may be the same as HD₂, described above.

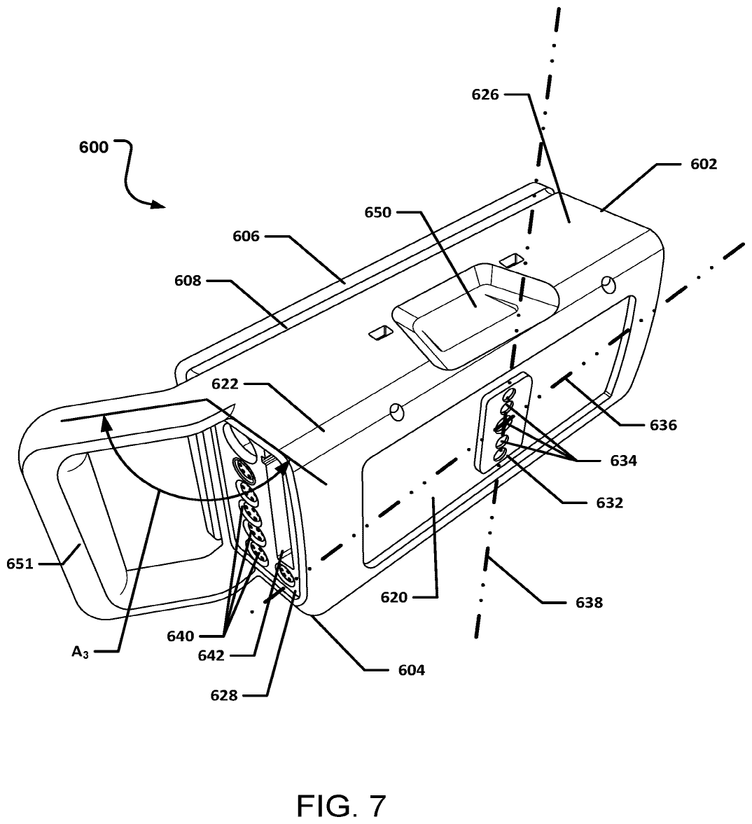

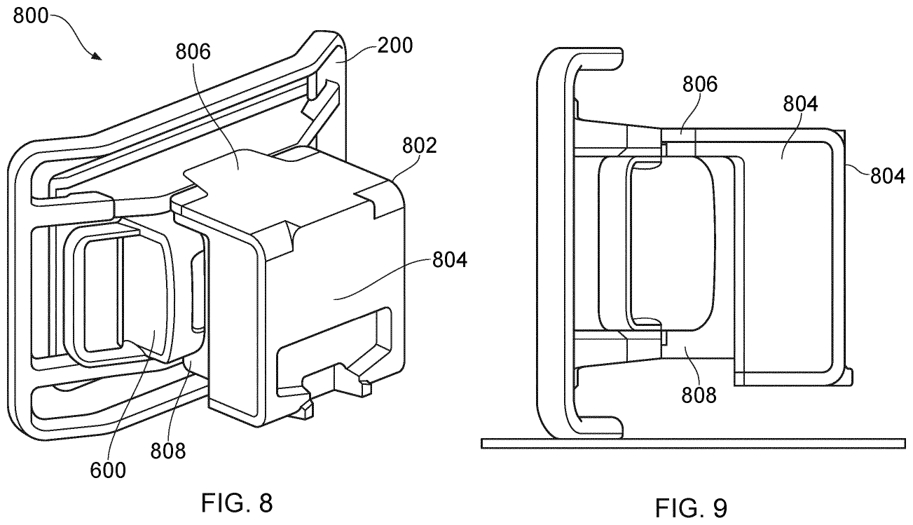

Referring now to FIG. 6 and FIG. 7, a first monitor 600 is depicted as one example. In a particular embodiment, the first monitor 600 may be the first monitor 120 described above in conjunction with the system illustrated in FIG. 1. FIG. 6 and FIG. 7 show that the first monitor 600 may include a housing 602 that may have a rear housing portion 604 and a front housing portion 606. The first monitor 600 may include an engagement groove 608 that may circumscribe the housing 602 between the rear housing portion 604 and the front housing portion 606. In a particular aspect, the engagement groove 608 may be sized and shaped to receive the elongated protrusions 222, 224 (FIG. 3) formed on the side walls 216, 218 (FIG. 3) of the docking slot 210 (FIG. 3) of the second monitor 200 (FIG. 3).

FIG. 6 shows that the front portion 606 of the housing 602 of the first monitor 600 may include a front face 610 and a user interface 612 may be incorporated into, or disposed in, the front face 610 of the front portion 606 of the housing 602. The user interface 612 may be a display, a touch display, or a combination thereof.

As illustrated in FIG. 6 and FIG. 7, the rear housing portion 604 of the first monitor 600 may include a back plate 620. An outer wall 622 may extend from the back plate 620. In particular, the outer wall 622 may extend from an outer perimeter of the back plate 620 in a direction that may be forward relative to the back plate 620 and substantially perpendicular to the back plate 620. The outer wall 622 of the rear housing portion 604 may include a lower wall portion 624 that may extend in a direction that may be substantially perpendicular to the back plate 620. The outer wall 622 of the rear housing portion 604 may also include an upper wall portion 626 opposite the lower wall portion 624 and spaced therefrom. The upper wall portion 626 of the outer wall 622 of the rear housing portion 604 may extend in a direction away from the back plate 620 that may be substantially perpendicular to the back plate 620 and substantially parallel to the lower wall portion 624.

As shown in FIG. 6 and FIG. 7, the outer wall 622 of the rear housing portion 604 may also include a left wall portion 628 that may extend between the lower wall portion 624 and the upper wall portion 626 of the outer wall 622 of the rear housing portion 604. The left wall portion 628 may extend from the back plate 620 in a direction that may be substantially perpendicular to the back plate 620. The left wall portion 628 may also be substantially perpendicular to the lower wall portion 624 and the upper wall portion 626. The outer wall 622 of the rear housing portion 604 may also include a right wall portion 630 that may extend between the lower wall portion 624 and the upper wall portion 626 of the outer wall 622 of the rear housing portion 604 opposite and spaced from the left wall portion 628. The right wall portion 630 may extend from the back plate 620 in a direction that may be substantially perpendicular to the back plate 620. The right wall portion 630 may also be substantially perpendicular to the lower wall portion 624 and the upper wall portion 626.

FIG. 7 further shows that the back plate 620 of the rear housing portion 604 may be formed with a mounting alignment plate 632. The mounting alignment plate 632 may include a plurality of ports 634 within the mounting alignment plate 632. As shown in FIG. 7, the mounting alignment plate 632 and the ports 634 formed therein are symmetrical about a horizontal axis 636 passing through a center of the mounting alignment plate 632. Further, the mounting alignment plate 632 and the port 634 formed therein are symmetrical about a vertical axis 638 passing through a center of the mounting alignment plate 632.

The left wall portion 628 of the outer wall 622 of the rear housing portion 602 may include a plurality of connector ports 640 and one or more patient monitoring ports 642. One or more cables may be connected to the first monitor 600 via the ports 640, 642 to allow the monitor 600 to receive power and to receive, consolidate, and display patient information and data from one or more physiological sensors. The patient information, or physiological information received from the physiological sensors, may include electrocardiograph (ECG) data, respiratory rate, noninvasive blood pressure (NIBP), invasive blood pressure (IBP) (systolic, diastolic, and mean), body temperature, pulse oximetry (SpO2), mixed venous oxygenation (SvO2), cardiac output, end-tidal carbon dioxide (ETCO2), intracranial pressure, airway gas concentrations, or a combination thereof.

As shown in FIG. 6, the right wall portion 630 of the outer wall 622 of the rear housing portion 604 may be formed with a docking connector plate 644. A docking connector 646 may be formed within the docking connector plate 644. As described in greater detail below, the docking connector plate 644 may be sized and shaped to fit into the docking connector recess 221 (FIG. 2) formed in the second monitor 200 (FIG. 2). FIG. 6 and FIG. 7 further show that the upper wall portion 626 of the outer wall 622 of the rear housing portion 604 of the housing 602 may be formed with an upper interlocking recess 650. The lower wall portion 624 may also include a similarly sized and shaped lower interlocking recess (not shown). As described in greater detail below, the interlocking recesses 650 of the rear housing portion 604 may receive and engage corresponding features formed on a monitor mount, described below.

As further illustrated in FIG. 6 and FIG. 7, the first monitor 600 may also include a handle 651 that may extend from the right wall portion 630 of the outer wall 622 of the rear housing 604 at an angle, A₃, with respect to the face of the right wall portion 630. In a particular aspect, A₃ may greater than or equal to 95°. In another aspect, A₃ may be greater than or equal to 100°, such as greater than or equal to 105°, greater than or equal to 110°, greater than or equal to 115°, greater than or equal to 120°, or greater than or equal to 125°. In still another aspect, A₃ may be less than or equal to 150°, such as less than or equal to 145°, less than or equal to 140°, less than or equal to 135°, or less than or equal to 130°. In yet another aspect, A₃ may be within a range between, and including, the minimum and maximum values of A₃ described herein.

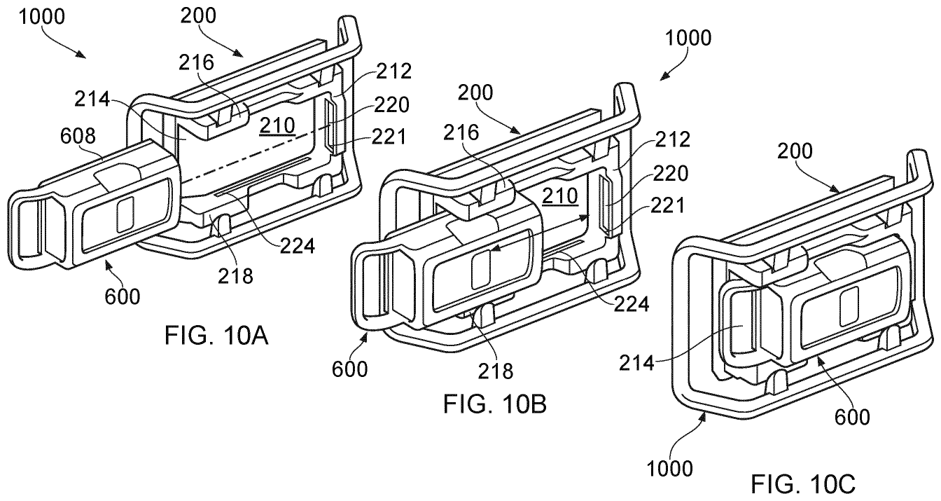

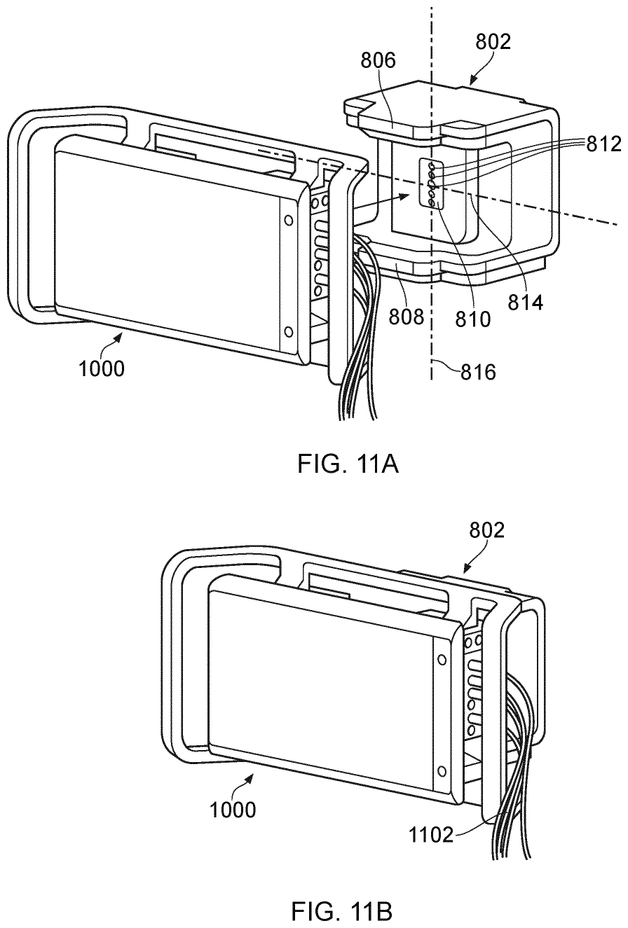

Referring now to FIG. 8 and FIG. 9, a patient monitoring assembly 800 is shown. The patient monitoring assembly 800 may include the first monitor 600, the second monitor 200, and a monitor mount 802.

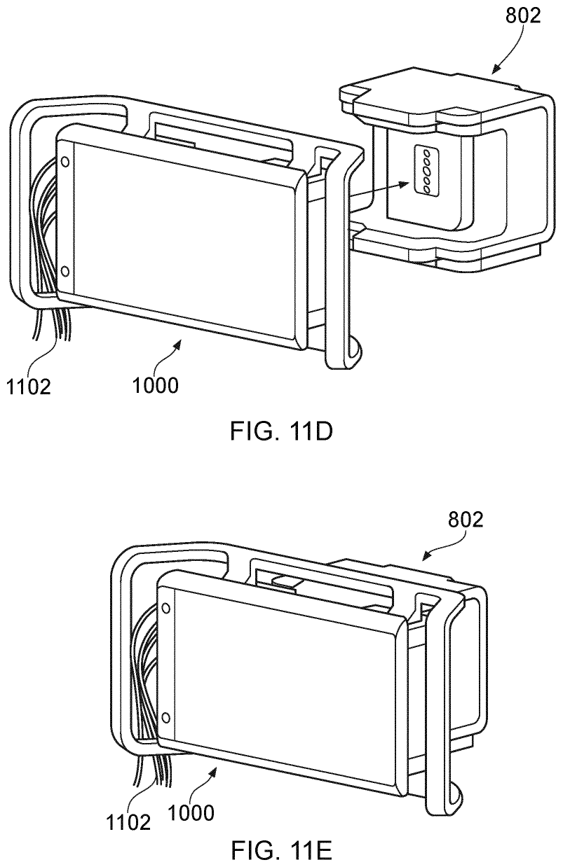

As illustrated in FIG. 8 and FIG. 9, the monitor mount 802 may include a housing 804. The housing 804 of the monitor mount 802 may have an upper latch 806 and a lower latch 808 that is space a distance from the upper latch mechanism 806. Further, the upper latch 806 may be parallel to the lower latch 808. Referring to FIGS. 11A, 11B, the monitor mount 802 may include a mounting alignment recess 810. The mounting alignment recess 810 may include a plurality of connectors 812 within the mounting alignment recess 810. As shown in FIGS. 11A, 11B, the mounting alignment recess 810 and the connectors 812 within the mounting alignment recess 810 are symmetrical about a horizontal axis 814 passing through a center of the mounting alignment recess 802. Further, the mounting alignment recess 810 and the connectors 812 within the mounting alignment recess 810 are symmetrical about a vertical axis 816 passing through a center of the mounting alignment recess 810.

In another aspect, the upper latch 806 and the lower latch 808 may be symmetrical about the horizontal axis 814 passing through the center of the mounting alignment recess 802. Additionally, the upper latch 806 and the lower latch 808 may be symmetrical about the vertical axis 816 passing through the center of the mounting alignment recess 802. In a particular aspect, the mounting alignment recess 802 may be sized and shaped to receive the mounting alignment plate 632 (FIG. 7) formed on the back plate 620 (FIG. 7) of the rear portion 604 (FIG. 7) of the housing 602 (FIG. 7) of the first monitor 600 (FIG. 7). In a particular aspect, the upper latch 806 and the lower latch 808 may be sized and shaped to fit into the first latch mechanism 230 (FIG. 3) within the first sidewall 216 (FIG. 3) of the docking slot 210 (FIG. 3) of the second monitor 200 and the second latch mechanism 232 (FIG. 3) within the second sidewall 218 (FIG. 3) of the docking slot 210 (FIG. 3) of the second monitor 200 (FIG. 3).

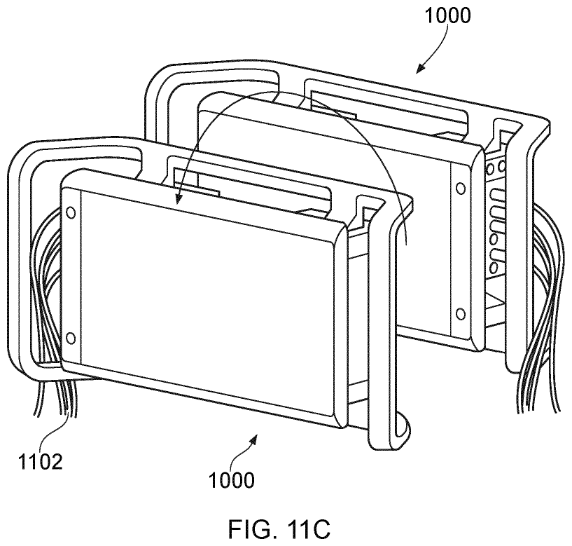

Referring now to FIG. 10A, FIGS. 10B, and 10C, a dual monitor assembly 1000 is shown and generally designated 1000. The dual monitor assembly 1000 may be formed by sliding the first monitor 600 into the docking slot 210 on the second monitor 200. As illustrated in FIG. 10A, when sliding the first monitor 600 into the docking slot 210, the first monitor 600 may enter the docking slot 210 via the open end 214 of the docking slot 210. The engagement groove 608 of the first monitor 600 may slide over and engage the elongated protrusions 222, 224 that may extend from, and at least partially along, the side walls 216, 218 of the docking slot 210.

As shown in FIG. 10B, the first monitor 600 may slide toward the closed end 212 of the docking slot 210 until the docking connector plate 644 of the first monitor 600 slides into the docking connector recess 221 within the closed end 212 of the docking slot 210 of the second monitor 200. The first monitor 600 may be fully engaged with the docking slot 210 of the second monitor 20, as depicted in FIG. 100, when the docking connector 646 within the docking connector plate 644 of the first monitor 600 is fully engaged with the docking connector 220 within the docking connector recess 221 within the closed end 212 of the docking slot 210 of the second monitor 200. In this configuration, the first monitor 600 may transmit all off the data that it receives from one or more physiologic sensors directly to the second monitor 200 via the docking connectors 220, 646.

FIG. 11A through 11E illustrate a patient monitoring system that includes the dual monitor assembly 1000 and the monitor mount 802. As shown, in FIG. 11A and FIG. 11B the dual monitor assembly 1000 may be formed as described above and then, the dual monitor assembly 1000 may be engaged with the monitor mount 802 by moving the dual monitor assembly 1000 into the monitor mount 802 so that the latches 806, 808 on the monitor mount 802 slide into and engage the latch mechanisms 230, 232 on the second monitor 200.

As shown in FIG. 11A, the dual monitor assembly 1000 may be oriented so that the upper latch 806 on the monitor mount 802 fits into and engages the first latch mechanism 230 on the second monitor 200 and the lower latch 806 on the monitor mount 802 fits into and engages the second latch mechanism 232 on the second monitor 200. Further, as the dual monitor assembly 1000 is engaged with the monitor mount 802, the mounting alignment plate 632 of the first monitor 600 may slide into and engage the mounting alignment recess 810 of the monitor mount 802 and the connectors 812 within the mounting alignment recess 810 of the monitor mount 802 may engage the ports 634 within the mounting alignment plate 632 of the first monitor 600. In this orientation, as depicted in FIG. 11B, a plurality of cables 1102 engaged with, and extending from, the first monitor 600 within the dual monitor assembly 1000 may protrude from a right side of the patient monitoring system.

If a user determines that it would be more appropriate for the cables 1102 to protrude from the left side of the patient monitoring system, the user may disengage the dual monitor assembly 1000 from the monitor mount 802 by grasping the handles 270, 290 on the second monitor 200 and pulling the dual monitor assembly 1000 away from, and out of, the monitor mount 802, e.g., toward the user. Thereafter, the user may rotate the dual monitor assembly 1000 counter-clockwise 180° so that the cables 1102 are hanging down the left side of the dual monitor assembly 1000, as shown in FIG. 11C.

Then, the user may re-engage the dual monitor assembly 1000 with the monitor mount 802 by moving the dual monitor assembly 1000 into the monitor mount 802 so that the latches 806, 808 on the monitor mount 802 slide into and engage the latch mechanisms 230, 232 on the second monitor 200. In this orientation, due to the symmetry of the latches 806, 808 and the latch mechanism, the dual monitor assembly 1000 may be oriented so that the upper latch 806 on the monitor mount 802 fits into and engages the second latch mechanism 232 on the second monitor 200 and the lower latch 806 on the monitor mount 802 fits into and engages the first latch mechanism 230 on the second monitor 200.

Further, due to the symmetry of the mounting alignment plate 632 of the first monitor 600, the symmetry of the ports 634 within the mounting alignment plate 632 of the first monitor 600, the symmetry of the mounting alignment recess 810, and the symmetry of the connectors 812 within the mounting alignment recess 810, the dual monitor assembly 1000 may be engaged with the monitor mount 802, so that the mounting alignment plate 632 of the first monitor 600 slides into and engages the mounting alignment recess 810 of the monitor mount 802—even though the dual monitor assembly 1000 has been rotated 180° from the orientation illustrated in FIG. 11A and FIG. 11B. In this orientation, the connectors 812 within the mounting alignment recess 810 of the monitor mount 802 may engage the ports 634 within the mounting alignment plate 632 of the first monitor 600. Moreover, in this orientation, as depicted in FIG. 11E, the plurality of cables 1102 engaged with, and extending from, the first monitor 600 within the dual monitor assembly 1000 may protrude from a left side of the patient monitoring system 1100. It can be appreciated that the dual monitor assembly 1000 may be moved from the first orientation to the second orientation without removing any of the cables 1102 from the first monitor 600 within the dual monitor assembly 1000. Further, it can be appreciated that the second monitor 200 may include the proper switching capability to orient the information presented at the user interface 208 so that it is readable by a user in either orientation relative to the monitor mount 802. In other words, the display image may flip as the dual monitor assembly 1000 is moved between the first orientation and the second orientation. This functionality can be provided using several existing solutions, such as an accelerometer and display software that changes the orientation of the screen based on its orientation.

Although various embodiments have been described above, these are to be regarded as merely examples. Accordingly, those of ordinary skill in the art will recognize that various changes and modifications of the examples described herein can be made without departing from the spirit and scope of the present disclosure. For example, any feature of any particular portion, embodiment or modification of the monitors 120, 140, 200, 400, 500, 600 may be included or omitted from any of the other portions, embodiments or modifications of the monitors 120, 140, 200, 400, 500, 600. Any feature of any particular portion, embodiment or modification of the monitor mount 160 may be included or omitted from any of the other portions, embodiments or modifications of the monitor mount 160.

In a particular aspect, when the dual monitor assembly 1000 is engaged with the monitor mount 802, as illustrated in FIG. 11B or FIG. 11E, a portion of each latch 806, 808 may fit into and engage the interlocking recesses 650 formed on the rear portion 604 of the housing 602 of the first monitor 600. This engagement may prevent the first monitor 600 from being withdrawn, or disengaged, from the docking slot 210 of the second monitor 200. Further, in the configurations shown in FIG. 11B or FIG. 11E, power, data, or a combination thereof may be transmitted to the dual monitor assembly 1000 via the connectors 812 and the ports 634. The power and data may be supplied to the first monitor 200 and the second monitor 600 as necessary.

Further, it is noted that the present disclosure may be implemented as any combination of a system, an integrated circuit, and a computer program on a non-transitory computer readable recording medium. The processor and any other parts of the computing system may be implemented as Integrated Circuits (IC), Application-Specific Integrated Circuits (ASIC), or Large Scale Integrated circuits (LSI), system LSI, super LSI, or ultra LSI components which perform a part or all of the functions of the computing system.

Each of the parts of the present disclosure can be implemented using many single-function components, or can be one component integrated using the technologies described above. The circuits may also be implemented as a specifically programmed general purpose processor, CPU, a specialized microprocessor such as Digital Signal Processor that can be directed by program instructions on a memory, a Field Programmable Gate Array (FPGA) that can be programmed after manufacturing, or a reconfigurable processor. Some or all of the functions may be implemented by such a processor while some or all of the functions may be implemented by circuitry in any of the forms discussed above.

The present disclosure may be implemented as a non-transitory computer-readable recording medium having recorded thereon a program embodying methods/algorithms for instructing the processor to perform the methods/algorithms. The non-transitory computer-readable recording medium can be, for example, a CD-ROM, DVD, Blu-ray disc, or an electronic memory device.

Each of the elements of the present disclosure may be configured by implementing dedicated hardware or a software program on a memory controlling a processor to perform the functions of any of the components or combinations thereof. Any of the components may be implemented as a CPU or other processor reading and executing a software program from a recording medium such as a hard disk or a semiconductor memory.

It is also contemplated that the implementation of the components of the present disclosure can be done with any newly arising technology that may replace any of the above implementation technologies.

1-29. (canceled)

30. A patient monitoring system, comprising:
a monitor mount;
a first monitor including a first user interface, a plurality of ports for receiving a plurality of cables from a plurality of physiologic sensors; and
a second monitor including a second user display, wherein:
the second monitor is adapted to receive and engage the first monitor to form a dual monitor assembly, and
the monitor mount is adapted to receive the dual monitor assembly in a first orientation in which the plurality of cables protrude from a first side of the dual monitor assembly and a second orientation opposite the first orientation in which the plurality of cables protrude from a second side of the dual monitor assembly.

31. The system of claim 30, wherein the second monitor includes a housing formed with a docking slot sized and shaped to receive the first monitor.

32. The system of claim 31, wherein the first monitor is adapted to slide into and engage the docking slot of the second monitor.

33. The system of claim 30, wherein the monitor mount comprises a mounting alignment recess extending into the monitor mount and configured to engage a portion of the first monitor.

34. The system of claim 33, wherein the mounting alignment recess comprises a plurality of connectors.

35. The system of claim 33, wherein the mounting alignment recess is symmetrical about a horizontal axis passing through a center of the mounting alignment recess.

36. The system of claim 33, wherein the first monitor comprises a mounting alignment plate extending from the first monitor and configured to fit into and engage the mounting alignment recess of the monitor mount.

37. The system of claim 30, wherein the dual monitor assembly is movable between the first orientation and the second orientation without removing any of the plurality of cables from the first monitor.

38. A patient monitor, comprising:
a housing having a docking slot formed therein, wherein the docking slot is sized and shaped to receive another patient monitor therein; and
a handle assembly extending from the housing, wherein the handle assembly comprises a first handle and a second handle that form a base adapted to support the patient monitor in an upright position.

39. The patient monitor of claim 38, wherein the handle assembly comprises a width, W, measured between a front of the handle assembly and a back of the handle assembly and W is greater than or equal to 7.00 cm.

40. The patient monitor of claim 38, wherein the handle assembly comprises a first transverse member and a second transverse member opposite the first transverse member and the first handle the second handle extend from the first transverse member and the second transverse member.

41. The patient monitor of claim 38, wherein each handle comprises a grip portion and each grip portion is spaced a handle distance, HD, the housing of the monitor to allow the user to wrap their fingers around the grip portion.

42. The patient monitor of claim 41, wherein HD is greater than or equal to 2.50 centimeters (cm).

43. The patient monitor of claim 38, wherein the handle assembly is symmetrical about a horizontal axis.

44. The patient monitor of claim 38, wherein the handle assembly is symmetrical about a vertical axis.

45. A method of operating a patient monitoring system, the method comprising:
engaging a first monitor with a second monitor to form a dual monitor assembly, wherein a plurality of physiologic sensor cables extend from the first monitor;
engaging the dual monitor assembly with a monitor mount in a first orientation;
disengaging the dual monitor assembly for the first orientation; and
engaging the dual monitor assembly with the monitor mount in a second orientation opposite the first orientation.

46. The method of claim 45, wherein dual monitor assembly is adapted to be moved from the first orientation to the second orientation without removing any of the plurality of physiologic sensor cables from the first monitor.

47. The method of claim 45, wherein the second orientation is 180° from the first orientation.

48. The method of claim 45, wherein in the first orientation, the plurality of physiologic sensor cables protrude from a first side of the dual monitor assembly.

49. The method of claim 48, wherein in the first orientation, the plurality of physiologic sensor cables protrude from a second side of the dual monitor assembly opposite the first side.
